(19) United States
(12) Patent Application Publication
(10) Pub. No.: US 2025/0170305 A1
(43) Pub. Date: May 29, 2025

(54) TRANSPARENT SCLERA-BASED CORNEAL REPAIR MATERIAL, AND PREPARATION METHOD AND USE THEREOF

(71) Applicant: EYE INSTITUTE OF SHANDONG FIRST MEDICAL UNIVERSITY (SHANDONG EYE INSTITUTE, QINGDAO EYE HOSPITAL OF, Shandong (CN)

(72) Inventors: Weiyun SHI, Shangdong (CN); Qingjun ZHOU, Shandong (CN); Long ZHAO, Shandong (CN); Xiuli SUN, Shandong (CN); Zhen SHI, Shandong (CN)

(21) Appl. No.: 18/262,692

(22) PCT Filed: Jan. 9, 2023
(86) PCT No.: PCT/CN2023/071153

§ 371 Date: Jul. 24, 2023

(30) Foreign Application Priority Data
Aug. 19, 2022 (CN) 202211003956.8

(51) Int. Cl. A61L 27/36

(52) U.S. Cl. CPC A61L 27/3691; A61L 27/3604; A61L 27/3675; A61L 2430/16

(57) ABSTRACT

A transparent sclera-based corneal repair material, and a preparation method and use thereof, belonging to the technical field of medical repair materials are disclosed. The sclera, a natural biological material that is convenient to obtain, is used as a source of the corneal repair material. The opaque sclera is hyalinized by compressing. Carboxyl groups on a scleral fibrin are activated by cross-linking, such that amino groups of the sclera combine with the carboxyl groups to form an amide bond. The amide bond belongs to a covalent bond and has a desirable stability, thereby maintaining a transparency of the sclera after the compressing. Therefore, the transparent sclera can be used as a transparent corneal tissue repair material.

### CROSS REFERENCE TO RELATED APPLICATION

The present application claims priority to the Chinese Patent Application No. CN202211003956.8, filed with the China National Intellectual Property Administration (CNIPA) on Aug. 19, 2022, and entitled “TRANSPARENT SCLERA-BASED CORNEAL REPAIR MATERIAL, AND PREPARATION METHOD AND USE THEREOF”, which is incorporated herein by reference in its entirety.

### TECHNICAL FIELD

The present disclosure relates to the technical field of medical repair materials, in particular to a transparent sclera-based corneal repair material, and a preparation method and use thereof.

### BACKGROUND

Corneal blindness is the fourth leading cause of blindness worldwide. There are about 10 million corneal blindness patients in the world, but only about 18,500 patients are capable of receiving keratoplasty annually. So far, for patients with severe corneal damage, the most common and effective way to improve visual function is still keratoplasty. This can ease the economic pressure on the country and improve the quality of life for patients. However, since the number of corneas donated by donors is far less than the demand for keratoplasty, it is necessary to develop new materials for corneal defect repair.

The cornea does not contain vascular tissues inside, which make the cornea have desirable optical penetration properties. Sutures are required during keratoplasty, such that repair materials need to have a certain suture-resistant strength. In other words, the repair materials need to show excellent biomechanical properties. After the keratoplasty, donor materials need to be epithelialized in vivo to complete the repair of biological tissues. Therefore, corneal repair materials require desirable biocompatibility. Due to different cases, these corneal repair materials also need to realize size controllability of the materials during the preparation.

The current corneal repair materials mainly include three categories. The first is that porcine cornea processed by decellularization is used as a substitute material for lamellar keratoplasty. Such a material has the disadvantage of heterogeneous origin. The second is biomimetic biomaterials prepared from the materials such as collagen, silk fibroin, alginate, or chitosan. This type of material has a poor biomechanical strength. The third is the artificial cornea made of polymethacrylate. This material has a narrow scope of application and is only suitable for patients with severe corneal damage.

Sclera is a natural biological material. From the source of development, both the sclera and cornea come from mesoderm. Moreover, both the sclera and cornea are derived from neural crest cells. The sclera is a collagen-rich extracellular matrix and has a collagen type similar to that of the cornea. From an ultrastructural point of view, like the cornea, the sclera is rich in transverse and longitudinal fibers. However, the sclera has characteristic oblique fibers that render the sclera opaque. As a result, sclera cannot currently be used as a corneal repair material.

### SUMMARY

An objective of the present disclosure is to provide a transparent sclera-based corneal repair material, and a preparation method and use thereof. The corneal repair material is a type of transparent sclera with desirable transparency and excellent biocompatibility.

To achieve the above objective, the present disclosure provides the following technical solutions:

The present disclosure provides a preparation method of a transparent sclera-based corneal repair material, including the following steps:
conducting pretreatment and decellularization on pig eye sclera in sequence to obtain a decellularized sclera section;
compressing the decellularized sclera section, and drying an obtained product until transparent to obtain a sclera sheet; and
subjecting the sclera sheet to cross-linking in a cross-linking agent solution, and immersing in water to obtain the transparent sclera-based corneal repair material.

Preferably, the pretreatment includes removal of uvea and accessory tissues, sectioning, and cleaning that are conducted in sequence.

Preferably, a sclera section obtained by the sectioning has a thickness of 350 μm to 500 μm.

Preferably, a reagent for the decellularization is a mixture II of Triton X-100 and a super nuclease; the Triton X-100 has a mass concentration of 1% to 3% in the mixture II; and the super nuclease has a concentration of 2,000 u/mL in the mixture II.

Preferably, the compressing is conducted at 10 MPa to 12 MPa for 48 h to 50 h.

Preferably, the compressing is conducted at a radian of 43° to 44°.

Preferably, the drying is conducted at 20° C. to 35° C. for 24 h to 26 h.

Preferably, a cross-linking agent in the cross-linking agent solution is 4-(4,6-dimethoxy-1,3,5-triazin-2-yl)-4-methylmorpholinium chloride (DMTMM); the cross-linking agent solution has a concentration of 30 wt %; and the cross-linking is conducted for 4.5 h to 5 h.

The present disclosure further provides a transparent sclera-based corneal repair material prepared by the preparation method.

The present disclosure further provides use of the transparent sclera-based corneal repair material in corneal repair.

The present disclosure provides a preparation method of a transparent sclera-based corneal repair material. In the present disclosure, the sclera, a natural biological material that is convenient to obtain, is used as a source of the corneal repair material. The opaque sclera is compressed into a sheet, such that the sclera has a reduced fiber spacing, a denser tissue, and a more orderly arrangement of fibers in three directions. In this way, the light transmittance is increased, and the transparency is increased, thereby realizing hyalinization of the sclera. Carboxyl groups on a scleral fibrin is activated by cross-linking, such that amino groups of the sclera combine with the carboxyl groups to form an amide bond. The amide bond belongs to a covalent bond and has a desirable stability, thereby maintaining a transparency of the sclera after the compressing. Therefore, obtained transparent sclera can be used as a transparent corneal tissue repair material.

In the present disclosure, sclera is used as a raw material. Accordingly, the prepared corneal repair material has fiber structure, as well as physical and chemical properties and biological properties similar to those of a natural corneal tissue, and shows excellent optical properties. Moreover, the sclera is a natural biological material with desirable biocompatibility and tissue compatibility. After being coated with Matrigel, corneal epithelial cells can adhere to, proliferate, and secrete extracellular matrix on this repair material, making the material epithelialized and facilitating postoperative corneal epithelial recovery. Therefore, the material can be used as an alternative of the corneal donor for the repair and replacement of damaged corneal tissues in the medical field.

In the present disclosure, the corneal tissue repair material uses natural sclera as a raw material. The mechanical properties of the natural sclera are higher than those of the cornea. Therefore, the repair material has desirable biomechanical properties, is suture-resistant, and can be made into materials with controllable size and thickness according to specific injury cases.

In the present disclosure, the preparation method has a simple process, cheap and easy-to-obtain raw materials, and a low cost, which are beneficial to large-scale production.

### BRIEF DESCRIPTION OF THE DRAWINGS

FIG. 1 shows a photo of the decellularized sclera section prepared in Example 1;

FIG. 2 shows a photo of the corneal repair material prepared in Example 1;

FIG. 3 shows a photo of the corneal repair material prepared in Example 1 after implanting a Matrigel basilar membrane;

FIG. 4 shows a section view (×400) of hematoxylin-eosin (H&E) staining of the corneal repair material prepared in Example 1;

FIG. 5 shows a transmission electron microscopy (TEM) image of regular arrangement of three-directional fibers of the corneal repair material prepared in Example 1;

FIG. 6 shows a TEM image of regular arrangement of transverse fibers of the corneal repair material prepared in Example 1;

FIG. 7 shows a TEM image of regular arrangement of longitudinal fibers of the corneal repair material prepared in Example 1;

FIG. 8 shows a slit lamp photo of 3 d after lamellar sclera transplantation in rabbits in Use Test Example 1;

FIG. 9 shows a photo of epithelial healing of a corneal graft 3 d after lamellar sclera transplantation in rabbits in Use Test Example 1;

FIG. 10 shows a general photo of 3 d after penetrating sclera transplantation in rabbits in Use Test Example 2;

FIG. 11 shows a slit lamp photo of 3 d after penetrating sclera transplantation in rabbits in Use Test Example 2; and

FIG. 12 shows a photo of epithelial healing of a corneal graft 3 d after penetrating sclera transplantation in rabbits in Use Test Example 2.

### DETAILED DESCRIPTION OF THE EMBODIMENTS

The present disclosure provides a preparation method of a transparent sclera-based corneal repair material, including the following steps:
conducting pretreatment and decellularization on pig eye sclera in sequence to obtain a decellularized sclera section;
compressing the decellularized sclera section, and drying an obtained product until transparent to obtain a sclera sheet; and
subjecting the sclera sheet to cross-linking in a cross-linking agent solution, and immersing in water to obtain the transparent sclera-based corneal repair material.

In the present disclosure, unless otherwise specified, all raw materials required for preparation are commercially available products well known to persons skilled in the art.

In the present disclosure, all operations in the preparation method are conducted in a sterile ultra-clean environment. The PBS has a concentration of 0.02 M and a pH of 7.4.

In the present disclosure, pretreatment and decellularization are conducted on pig eye sclera in sequence to obtain a decellularized sclera section.

In the present disclosure, the pig eye sclera is preferably obtained from fresh eyeballs collected within 2 h, and the sclera is cleaned with sterile double distilled water. There is no special limitation on a cleaning process, which can be conducted according to the well-known process in the art.

In the present disclosure, the pretreatment includes preferably removal of uvea and accessory tissues, sectioning, and cleaning that are conducted in sequence. Preferably, the uvea and accessory tissues are surgically removed; more preferably, the uvea and accessory muscular tissues are removed from the inner and outer surfaces of the pig eye sclera. There is no special limitation on a removal process, which can be conducted according to the process well known in the art.

In the present disclosure, the inner and outer sides of the sclera are resected through the sectioning. Preferably, the sectioning is conducted using a Moria microkeratome and a trephine of a specified size, and sections of the resulting size and thickness are obtained. The sclera section has a thickness of preferably 350 μm to 500 μm and a diameter of 6.25 mm.

In the present disclosure, a cleaning solution for the cleaning is preferably a mixture I of Triton X-100 and PBS, and the Triton X-100 has a mass concentration of preferably 1% to 3%, more preferably 2% in the mixture I. The cleaning solution is preferably used after being sterilized under high temperature and high pressure. The cleanings is preferably conducted greater than or equal to 3 times for preferably 30 min each time and a total cleaning time of preferably 2 h to 2.5 h. There is no special limitation on a composition of the PBS, and commercially available products well known in the art can be used; in an example, the PBS has a concentration of 0.02 mol/L and a pH value of 7.4. There is no special limitation on a process of the sterilization under high temperature and high pressure, which can be conducted according to the process well known in the art.

In the present disclosure, after the pretreatment is completed, the sclera section is subjected to the decellularization. A reagent for the decellularization is preferably a mixture II of Triton X-100 and a super nuclease. A solvent used in the mixture II of the Triton X-100 and the super nuclease is preferably pure water. The Triton X-100 has a mass concentration of 1% to 3%, more preferably 2% in the mixture II. The super nuclease has a concentration of 2,000 u/mL in the mixture II. There is no special limitation on a dosage of the mixture II of the Triton X-100 and the super nuclease, as long as the decellularization is fully conducted.

In the present disclosure, the decellularization is conducted at preferably 37° C. for preferably 15 h to 17 h in preferably a shaker with a rotation speed of preferably 100 rpm. The scleral fibroblasts are removed through the decellularization, thereby alleviating a rejection reaction of the sclera sheet after being applied to living body transplantation.

In the present disclosure, after the decellularization is completed, the sclera section is preferably washed with PBS. There is no special limitation on a washing process, and the sclera section can be washed according to the well-known process in the art.

In the present disclosure, the decellularized sclera section is compressed, and an obtained product is dried until transparent to obtain a sclera sheet. The compressing is preferably conducted in a press. The compressing is conducted at preferably 10 MPa to 12 MPa for preferably 48 h to 50 h. The compressing is conducted at a radian of preferably 43° to 44° (same as that of the cornea), more preferably 43.5°. The fibrin arrangement of the sclera is more orderly by the compressing. After the fiber is ordered, the scattering of light after passing through the tissues is reduced, and the transmittance of light is increased.

In the present disclosure, the drying is preferably conducted under natural conditions at preferably 20° C. to 35° C. for preferably 24 h to 26 h. There is no special limitation on a degree of the drying to transparency, as long as a desired degree of transparency can be achieved.

In the present disclosure, the sclera sheet is subjected to cross-linking in a cross-linking agent solution, and immersed in water to obtain the transparent sclera-based corneal repair material.

In the present disclosure, a cross-linking agent in the cross-linking agent solution is preferably 4-(4,6-dimethoxy-1,3,5-triazin-2-yl)-4-methylmorpholinium chloride (DMTMM); the cross-linking agent solution has a concentration of preferably 30 wt %; and the cross-linking is conducted for preferably 4.5 h to 5 h. The cross-linking agent solution is preferably obtained by dissolving a DMTMM powder in an HCl solution with a pH value of 6.5. There is no special limitation on a dosage of the cross-linking agent solution, as long as the solution can completely infiltrate the sclera sheet.

In the present disclosure, the cross-linking agent can activate the carboxyl groups of scleral fibrin, and make the amino groups and carboxyl groups of sclera combine to form an amide bond. The amide bond is a covalent bond, which is more stable compared with the non-cross-linked amino and carboxyl groups, and can maintain the transparency of sclera after compressing. An amidation equation is: R—COOH+R′—NH₂→R—CO—NH—R′ (final structure, type I collagen)+H₂O.

In the present disclosure, a process of the immersing includes preferably: immersing the sclera sheet obtained by cross-linking into PBS or a 0.9 wt % sodium chloride aqueous solution until the immersing is completed. The PBS has a concentration of preferably 0.02 mol/L and a pH value of preferably 7.4.

The present disclosure further provides a transparent sclera-based corneal repair material prepared by the preparation method.

The present disclosure further provides use of the transparent sclera-based corneal repair material in corneal repair. In the present disclosure, there is no special limitation on a method of the use, and methods well known in the art can be used. In the present disclosure, preferably, the transparent sclera-based corneal repair material is coated with a Matrigel basilar membrane, so as to increase the biocompatibility of the tissues, thereby promoting the rapid repair of the corneal epithelium. There is no special limitation on a process of coating with the Matrigel basilar membrane, which can be conducted according to the process well known in the art. In an example, the process specifically includes: implanting the corneal repair material into a Matrigel basilar membrane solution with a concentration of 8 mg/mL (the Matrigel basilar membrane dissolved in a Gibco's serum-free medium), spreading, and drying in an oven at 37° C.; conducting immersing with PBS (with a concentration of 0.02 mol/L and a pH value of 7.4) to obtain a composite membrane. Preferably, the composite membrane is directly used for corneal repair.

The technical solutions of the present disclosure will be clearly and completely described below with reference to the examples of the present disclosure. Apparently, the described examples are merely a part rather than all of the examples of the present disclosure. All other embodiments obtained by a person of ordinary skill in the art based on the embodiments of the present disclosure without creative efforts shall fall within the protection scope of the present disclosure.

In the following examples, the PBS has a concentration of 0.02 mol/L and a pH value of 7.4.

### Example 1

1) fresh pig eye sclera within 2 h was taken, and uvea and accessory muscle tissues on the inner and outer surfaces of the sclera were removed; treated sclera was sectioned with a Moria microkeratome and a trephine of a specific size, to obtain a sclera section with a thickness of 500 μm and a diameter of 6.25 mm.

the sclera section was washed in a Triton X-100-PBS solution with a mass concentration of 2% for 2 h, 4 times, 30 min for each time; a resulting washed sclera section was placed in a mixture of 2% TritonX-100 and 2,000 u/mL super nuclease, and the subjected to decellularization in a shaker at 37° C. and 100 rpm for 17 h; a resulting product was washed again with PBS to obtain a decellularized sclera sheet;
the decellularized pig eye sclera sheet with a diameter of 6.25 mm and a thickness of 500 μm was compressed in a press at 10 MPa and a radian of 43.5° for 48 h, and then dried at a room temperature for 24 h to obtain a transparent sclera sheet; and
the sclera sheet was cross-linked in a 30 wt % cross-linking agent, DMTMM solution (a DMTMM powder dissolved in an HCl solution with a pH value of 6.5) for 5 h, and then immersed in the PBS to obtain a corneal repair material.

### Use Example 1

The corneal repair material prepared in Example 1 was placed in a petri dish, and 200 μL of a Matrigel basilar membrane solution with a concentration of 8 mg/mL (a Matrigel basilar membrane was dissolved in a Gibco's serum-free medium) was added dropwise into an upper surface of the material, spread with a pipette tip, dried in an oven at 37° C. for 2 h, and then immersed with PBS (with a concentration: 0.02 mol/L and a pH value: 7.4) to obtain a composite membrane. Note: since the Matrigel membrane was easy to gel at higher than 10° C., all operations were conducted on ice before drying in the oven.

### Morphological Observation

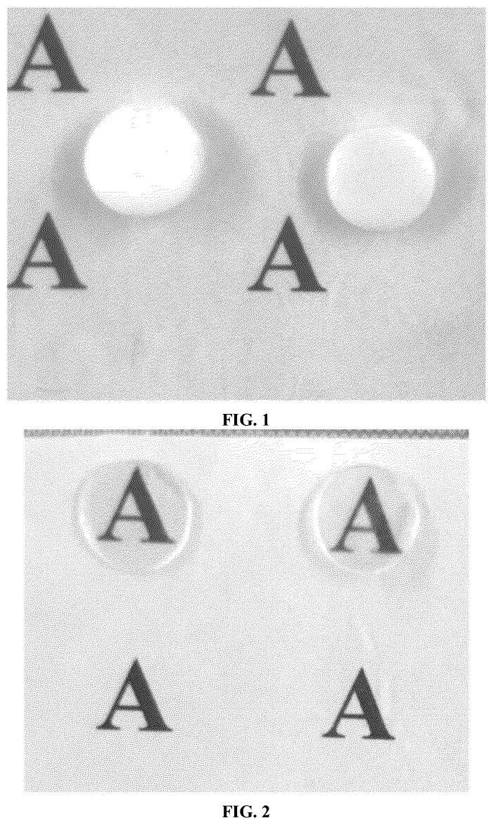

1) FIG. 1 showed a photo of the decellularized sclera section prepared in Example 1. This indicated that the sclera appeared opaque, porcelain white after decellularization.

2) FIG. 2 shows a photo of the corneal repair material prepared in Example 1. This indicated that the decellularized sclera section maintained high transparency after being compressed, dried, cross-linked, and immersed.

3) FIG. 3 showed a photo of the corneal repair material prepared in Example 1 after being implanted with the Matrigel basilar membrane. There was no obvious change in the transparency of the transparent sclera coated with the Matrigel basilar membrane. The results were shown in FIG. 3.

4) The corneal repair material prepared in Example 1 was stained with hematoxylin-eosin (H&E), specifically including:

1. Fixation: the corneal repair material prepared in Example 1 was fixated in a 10 wt % formaldehyde solution for 24 h.

2. Dehydration: an obtained fixated material was placed in an embedding box, rinsed in running water for 30 min, and dehydrated in an alcohol solution.

3. Transparency treatment: an obtained dehydrated material was placed in xylene to replace the alcohol to maintain the transparency of the material.

4. Wax dipping and embedding: the transparent material was placed in melted paraffin, and cooled after the paraffin completely covered the material, so as to achieve the embedding.

5. Slicing, unfolding, and baking: an obtained embedded material was cut into thin slices with a thickness of 3 μm in a microtome, the thin slices were put in water and unfolded, and placed on slides. The slides were dried in a thermostat at 60° C.

6. Dewaxing and dehydration: the slides were dewaxed in xylene, dehydrated in alcohol, and placed in distilled water.

7. Staining: the slices in distilled water were stained for 10 min in a hematoxylin aqueous solution, placed in 1 wt % hydrochloric acid for 3 s, and then placed in tap water for 5 min to conduct bluing; resulting bluish slices were dehydrated in 70% and 90% alcohol separately for 10 min, and then stained in an eosin staining solution for 5 min.

8. Dehydration and hyalinization: an obtained dyed material was dehydrated in pure alcohol, and then placed in xylene for hyalinization.

9. Sealing and observation: the slices were observed after adding resin on the glass slide and sealing with the cover glass.

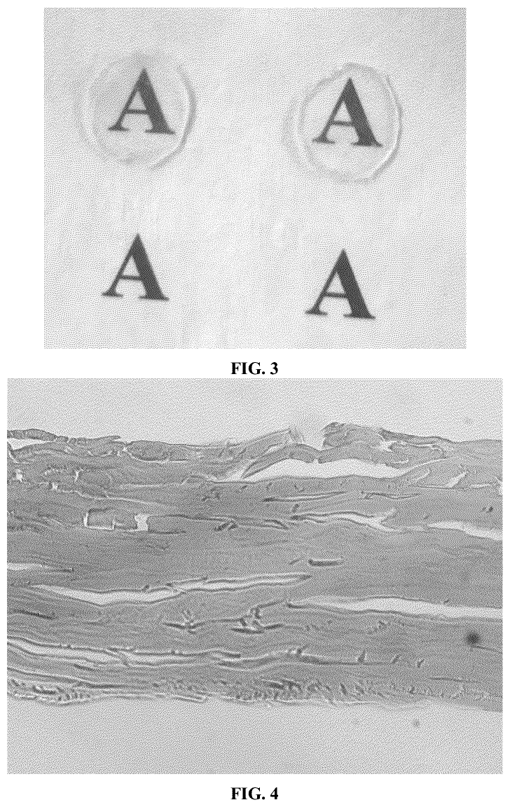

The stained slices (×400) obtained through the above steps 1 to 9 were shown in FIG. 4. It was seen from FIG. 4 that the transparent corneal repair material prepared by the method of the present disclosure had relatively regular and consistent fiber arrangement.

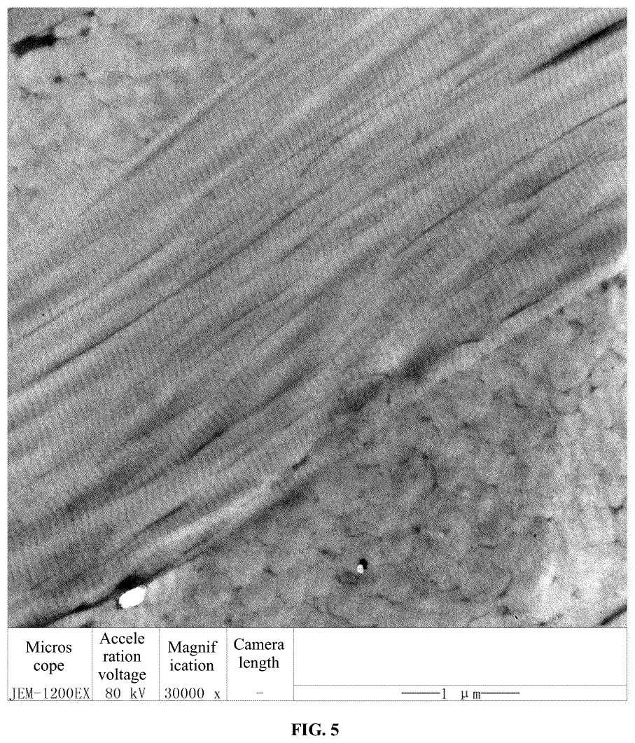

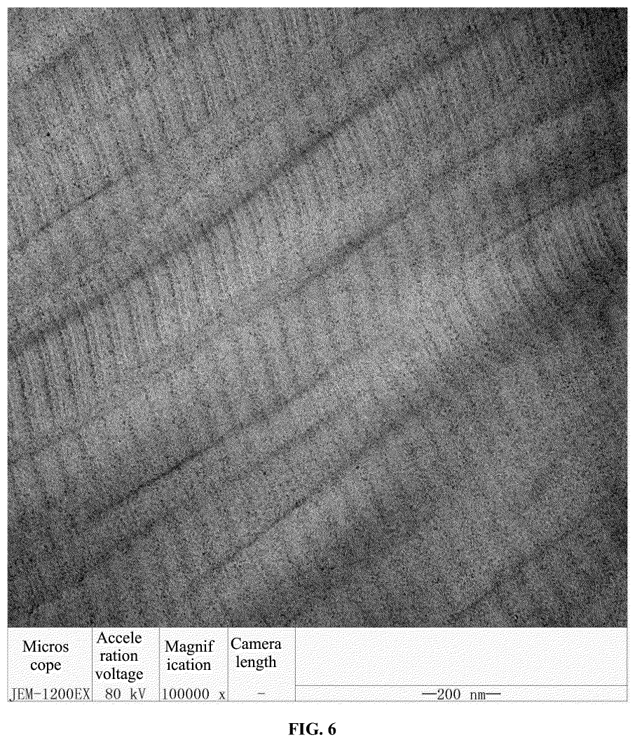

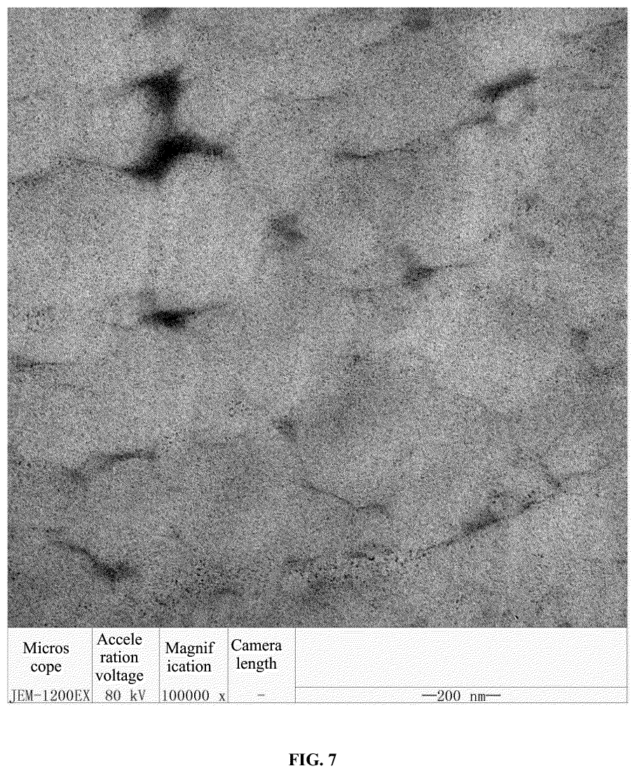

5) A TEM test was conducted on the corneal repair material prepared in Example 1, and the obtained results were shown in FIG. 5 to FIG. 7. From FIG. 5 to FIG. 7, it was seen that the hyalinized scleral tissue had tightly-arranged and orderly three-directional fibers, and a fiber spacing was significantly reduced.

### Use Test Example 1

The composite membrane prepared in Use Example 1 was used to repair the defect of rabbit cornea, and conduct lamellar keratoplasty in rabbits.

(1) Animal model preparation: healthy New Zealand white rabbits were anesthetized, and the corneal stroma with a diameter of 6.0 mm and a thickness of 200 μm was removed in a center of the cornea with a trephine to prepare a corneal stroma defect model.

(2) Defect repair: the corneal defect was filled with the transparent sclera material coated with the Matrigel basilar membrane prepared in Use Example 1, sutured intermittently with 10-0 nylon suture, and a thread knot was transferred to the deep corneal stroma. Note: the transplant was aligned with the planting bed.

(3) Rinsing: the cornea was rinsed with normal saline, and after binding with a corneal bandage mirror, the New Zealand rabbits were sent back to their breeding room.

(5) Postoperative care: the New Zealand white rabbits were smeared with Alcon® eye drops (tobramycin and dexamethasone eye drops) every morning and evening within seven days after the surgery.

### Evaluation of Results

1. General photo observation: 3 d after surgery, a repair effect of the transparent sclera material coated with the Matrigel basilar membrane prepared in Use Example 1 was observed by a slit lamp. The results were shown in FIG. 8. It was seen from FIG. 8 that in the rabbit cornea repaired by the repair material, after 3 d of the lamellar keratoplasty in the rabbit body, the material still remained relatively transparent, and the central pupil area was relatively clear.

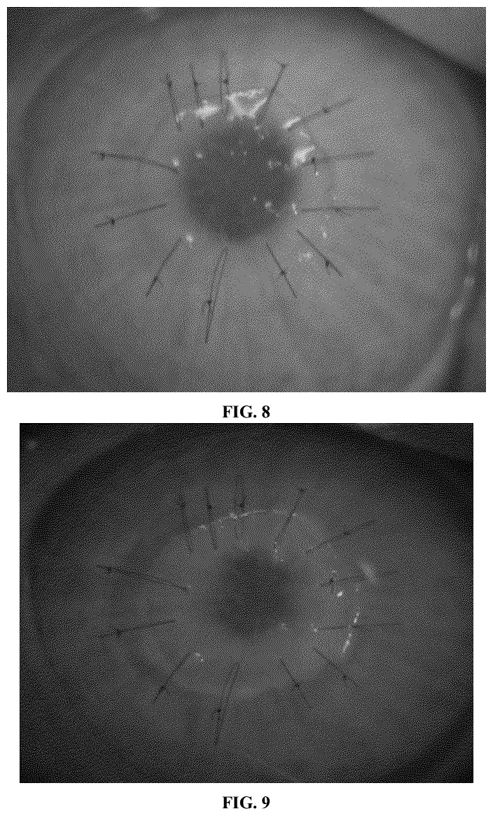

2. Observation of sodium fluorescein staining: 3 d after surgery, the cornea repaired with the transparent sclera repair material coated with the Matrigel basilar membrane prepared in Use Example 1 was stained with sodium fluorescein (the conjunctival sac at the outer ⅓ of the lower eyelid of the rabbit was lightly touched with a sodium fluorescein staining test paper to make the rabbit blink, such that the stain was evenly applied to the corneal surface of the rabbit). After the stained cornea was rinsed with normal saline, photographing and observation were conducted under cobalt blue light, and the results were shown in FIG. 9. As shown in FIG. 9, the rabbit corneal epithelium repaired by the repair material was completely repaired, and corneal epithelialization had been realized.

### Use Test Example 2

The composite membrane prepared in Use Example 1 was used to treat corneal perforation in rabbits, and conduct penetrating keratoplasty in rabbits.

(1) Animal model preparation: healthy New Zealand white rabbits were anesthetized, and a full-thickness cornea with a diameter of 2.75 mm was removed by a trephine in a center of the cornea to prepare a corneal perforation model.

(2) Defect repair: the corneal perforation was filled with the transparent sclera material coated with the Matrigel basilar membrane prepared in Use Example 1, sutured intermittently with 10-0 nylon suture, and a thread knot was transferred to the deep corneal stroma. Note: the transplant was aligned with the planting bed.

(3) Rinsing: the cornea was rinsed with normal saline, and after binding with a corneal bandage mirror, the New Zealand rabbits were sent back to their breeding room.

(5) Postoperative care: the New Zealand white rabbits were smeared with Alcon® eye drops (tobramycin and dexamethasone eye drops) every morning and evening within seven days after the surgery.

### Evaluation of Results

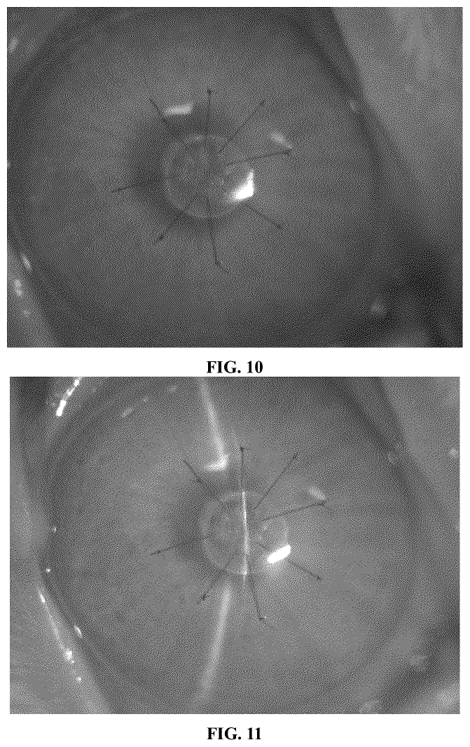

1) General photo observation: 3 d after surgery, a repair effect of the transparent sclera material coated with the Matrigel basilar membrane prepared in Use Example 1 was observed by a slit lamp. The results were shown in FIG. 10. It was seen from FIG. 10 that in the rabbit cornea repaired by the repair material, 3 d after the penetrating keratoplasty in rabbit body, the material still remained relatively transparent, and the central pupil area was relatively clear.

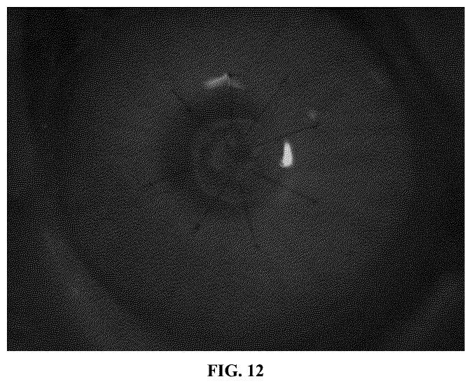

2) 3 d after the surgery, digital slit lamp inspection was conducted on the rabbit in vivo model, and the results were shown in FIG. 11. As shown in FIG. 11, the rabbit corneal perforation repaired by this repair material had an anterior chamber that was well formed.

3) Observation of sodium fluorescein staining: 3 d after surgery, the cornea repaired with the transparent sclera repair material coated with the Matrigel basilar membrane prepared in Use Example 1 was stained with sodium fluorescein (the conjunctival sac at the outer ⅓ of the lower eyelid of the rabbit was lightly touched with a sodium fluorescein staining test paper to make the rabbit blink, such that the stain was evenly applied to the corneal surface of the rabbit). After the stained cornea was rinsed with normal saline, photographing and observation were conducted under cobalt blue light, and the results were shown in FIG. 12. As shown in FIG. 12, the rabbit corneal epithelium repaired by the repair material was completely repaired, and corneal epithelialization had been realized.

The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

1. A preparation method of a transparent sclera-based corneal repair material, comprising the following steps:
conducting pretreatment and decellularization on pig eye sclera in sequence to obtain a decellularized sclera section;
compressing the decellularized sclera section, and drying an obtained product until transparent to obtain a transparent sclera sheet; and
subjecting the transparent sclera sheet to cross-linking in a cross-linking agent solution, and immersing in water to obtain the transparent sclera-based corneal repair material.

2. The preparation method according to claim 1, wherein the pretreatment comprises removal of uvea and accessory tissues, sectioning, and cleaning that are conducted in sequence.

3. The preparation method according to claim 2, wherein a sclera section obtained by the sectioning has a thickness of 350 μm to 500 μm.

4. The preparation method according to claim 3, wherein the sclera section obtained by the sectioning has a diameter of 6.25 mm.

5. The preparation method according to claim 2, wherein a cleaning solution for the cleaning is a mixture I of Triton X-100 and a PBS, and the Triton X-100 in the mixture I has a mass concentration of 1% to 3%; and the PBS has a concentration of 0.02 mol/L and a pH value of 7.4.

6. The preparation method according to claim 5, wherein the cleaning is conducted greater than or equal to 3 times for 30 min each time and a total cleaning time of 2 h to 2.5 h.

7. The preparation method according to claim 1, wherein a reagent for the decellularization is a mixture II of Triton X-100 and a super nuclease; the Triton X-100 has a mass concentration of 1% to 3% in the mixture II; and the super nuclease has a concentration of 2,000 u/mL in the mixture II.

8. The preparation method according to claim 7, wherein the Triton X-100 has a mass concentration of 2% in the mixture II of the Triton X-100 and the super nuclease.

9. The preparation method according to claim 1, wherein the decellularization is conducted at 37° C. for 15 h to 17 h in a shaker with a rotation speed of 100 rpm.

10. The preparation method according to claim 1, wherein the compressing is conducted at 10 MPa to 12 MPa for 48 h to 50 h.

11. The preparation method according to claim 1, wherein the compressing is conducted at a radian of 43° to 44°.

12. The preparation method according to claim 11, wherein the compressing is conducted at a radian of 43.5°.

13. The preparation method according to claim 1, wherein the drying is conducted at 20° C. to 35° C. for 24 h to 26 h.

14. The preparation method according to claim 1, wherein a cross-linking agent in the cross-linking agent solution is 4-(4,6-dimethoxy-1,3,5-triazin-2-yl)-4-methylmorpholinium chloride (DMTMM); the cross-linking agent solution has a concentration of 30 wt %; and the cross-linking is conducted for 4.5 h to 5 h.

15. A transparent sclera-based corneal repair material prepared by the preparation method according to claim 1.

16. A corneal repair material for repairing cornea, prepared from the transparent sclera-based corneal repair material according to claim 1.

17. The preparation method according to claim 7, wherein the decellularization is conducted at 37° C. for 15 h to 17 h in a shaker with a rotation speed of 100 rpm.

18. The preparation method according to claim 8, wherein the decellularization is conducted at 37° C. for 15 h to 17 h in a shaker with a rotation speed of 100 rpm.

19. The preparation method according to claim 10, wherein the compressing is conducted at a radian of 43° to 44°.

20. The preparation method according to claim 19, wherein the compressing is conducted at a radian of 43.5°.
